(19) United States
(12) Patent Application Publication
(10) Pub. No.: US 2004/0199014 A1
(43) Pub. Date: Oct. 7, 2004

(54) Method for producing 2-(hydroxyphenyl)-2-(alkoxyimino)-n-methylacetamide derivatives

(76) Inventors: Holger Weintritt, Langenfeld (DE); Reinhard Lantzsch, Wuppertal (DE); Thorsten Muh, Levekusen (DE)

Correspondence Address: Bayer CropScience, Patent Department, 100 Bayer Road, Pittsburgh, PA, 15205-9741, US

(21) Appl. No.: 10/485,809

(22) PCT Filed: Jul. 24, 2002
(86) PCT No.: PCT/EP02/08244

(30) Foreign Application Priority Data
Aug. 6, 2001 (DE) 101385757

(51) Int. Cl. C07C 251/48

(52) U.S. Cl. 564/147

(57) ABSTRACT

The invention relates to a novel process for preparing (2E)-2-(hydroxyphenyl)-2-(alkoxyimino)-N-methylacetamides.

The invention relates to processes for preparing 2-(hydroxyphenyl)-2-(alkoxyimino)-N-methylacetamide derivatives, to novel intermediates for their preparation and to processes for preparing these intermediates.

It is already known that dichlorobenzofuran-3-one is obtained by chlorination of benzofuran-3-one (compare, for example, Chem. Ber. 1912, p. 161). The reaction known from the prior art is carried out in a closed vessel and can therefore not be carried out on an industrial scale. Moreover, dichlorobenzofuran-3-one is obtained as byproduct in the preparation of 2-methoxybenzoyl chloride (cf. DE-A-2040186). However, a process for the preparation specifically of dichlorobenzofuran-3-one which can be practised on an industrial scale has hitherto not been disclosed. Ring opening of dichlorobenzofuran-3-one has likewise not been disclosed in the literature.

It is already known that 2-(2-hydroxyphenyl)-2-(methoxyimino)-N-methylacetamide (C) is obtained when methyl 2-(2-hydroxyphenyl)-2-(methoxyimino)acetate (A) or 1-benzofuran-2,3-dione 3-(O-methyloxime) (B) is reacted with methylamine (compare, for example, EP-A 398 692 or WO 95/24396):

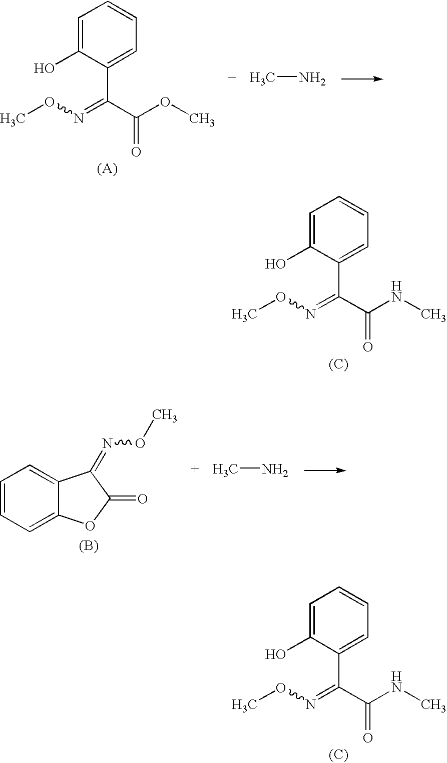

The disadvantage of these processes is that the starting materials are difficult to obtain.

WO 01/38294 describes two processes for preparing 2-(2-hydroxyphenyl)-2-(alkoxyimino)-N-methylacetamide derivatives starting from 2-(2-hydroxyphenyl)-2-(hydroxyimino)-N-methylacetamide or starting from 2-(2-hydroxyphenyl)-2-(methylimino)-N-methylacetamide.

It has now been found that 2-(hydroxyphenyl)-2-(alkoxyimino)-N-methylacetamide derivatives of the general formula (I)

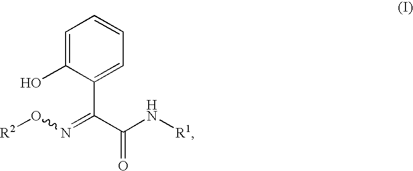

in which

R¹ and R² represent C₁-C₄-alkyl,

are obtained when

a) a compound of the formula (II)

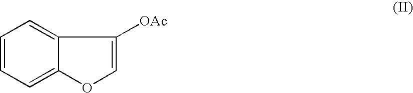

is reacted with chlorine gas, as appropriate in the presence of a diluent;

b) the resulting compound of the formula (III)

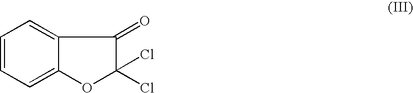

is reacted with an amine of the formula (IV),

R¹—NH₂ (IV)

in which

R¹ is as defined above

—or an acid addition complex thereof—,

if appropriate in the presence of a diluent and if appropriate in the presence of an acid acceptor, giving a compound of the formula (V);

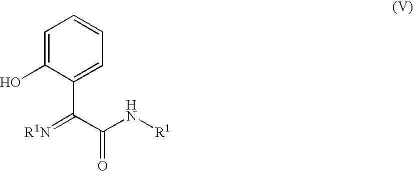

which is

c1) reacted with hydroxylamine—or an acid addition complex thereof—, if appropriate in the presence of a diluent and if appropriate in the presence of a buffer medium, giving compounds of the formula (VI);

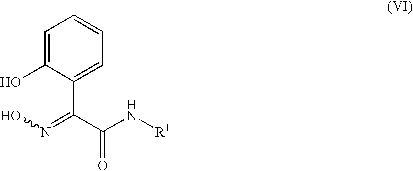

and

c2) reacted with an alkylating agent of the formula (VII),

R²—X (VII)

in which

R² is as defined above and

X represents halogen, —O—CO—O—R², —O—SO₂—R², or —O—SO₂—O—R², where R² is as defined above,

if appropriate in the presence of a diluent and if appropriate in the presence of an acid acceptor, if appropriate in the presence of a phase-transfer catalyst;

or

d) reacted with alkoxy amines of the formula (VIII)

R²—O—NH₂ (VIII),

in which

R² is as defined above

—or an acid addition complex thereof—,

if appropriate in the presence of a diluent and if appropriate in the presence of a buffer medium.

In the definition of R¹ and R², the saturated C₁-C₄-alkyl hydrocarbon chains are in each case straight-chain or branched. C₁-C₄-alkyl represents in particular methyl, ethyl, n- or i-propyl, n-, s- or i-butyl.

R¹ represents in particular methyl, ethyl or n-propyl.

R¹ particularly preferably represents methyl.

R² represents in particular methyl, ethyl or n-propyl.

R² particularly preferably represents methyl.

The general or preferred radical definitions listed above apply both to the end products of the formula (I) and, correspondingly, to the starting materials or intermediates required in each case for the preparation.

The specific radical definitions for these radicals given in the respective combinations or preferred combinations of radicals are, independently of the particular combination of radicals given, also replaced by any radical definitions of other preferred ranges.

If appropriate, the compounds according to the invention can be present as mixtures of different possible isomeric forms, in particular of stereoisomers, such as, for example, E and Z. What is claimed are both the E- and the Z-isomers and any mixtures of these isomers.

In the prior art, the chlorination of enole acetates is not disclosed. It is extremely surprising that in reaction step a) of the process according to the invention the chlorination of the enole acetate of the formula (II) takes place with removal of the acetate group. Ring opening of dichlorobenzofuran-3-one with amines has likewise not been disclosed in the literature.

The process according to the invention has a number of advantages. The process according to the invention is used, in particular, for preparing 2-(2-hydroxyphenyl)-2-(alkoxyimino)-N-methylacetamide derivatives, which are important intermediates for the preparation of pesticides (compare, for example, EP-A 398 692 and EP-A-937 050). The process according to the invention opens a new synthetic route to 2-(2-hydroxyphenyl)-2-(alkoxyimino)-N-methylacetamide derivatives which is characterized by the starting materials being readily available, by good yields of the individual reaction steps and by easy realization of the individual reaction steps on an industrial scale.

This novel process is shown in reaction scheme 1.

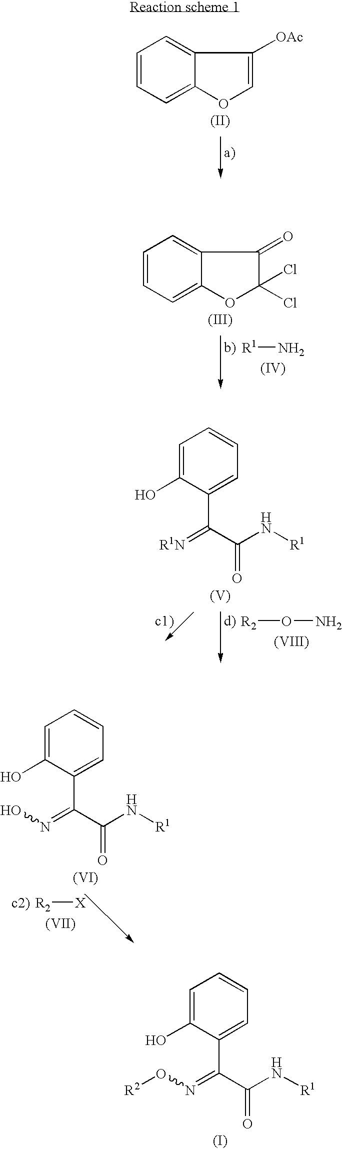

The compound of the formula (II) required as starting material for carrying out reaction step a) of the process according to the invention is already known and can be prepared by known methods (compare, for example, J. Chem. Soc. Perkin Trans 1, 1984, 1605-1612, WO 01/36405).

The chlorine furthermore required as starting material for carrying out reaction step a) of the process according to the invention is a chemical for synthesis which is commercially available. It can be used either as a gas or as an alcoholic or aqueous solution in a diluent.

The amines of the formula (IV) or their acid addition complexes furthermore required as starting materials for carrying out reaction step b) of the process according to the invention are chemicals for synthesis which are commercially available. They can be employed either, if appropriate, as a gas or as an alcoholic or aqueous solution. Preferred amines of the formula (IV) are ethylamine and methylamine. Particular preference is given to methylamine. Preferred acid addition complexes are the hydrochlorides, hydrogen sulphates or sulphates.

The hydroxylamine or its acid addition complexes furthermore required as starting materials for carrying out reaction step c1) of the process according to the invention are chemicals for synthesis which are commercially available. Preferred acid addition complexes are the hydrochlorides, hydrogen sulphates or sulphates.

Alkylating agents of the formula (VII) furthermore required as starting materials for carrying out reaction step c2) of the process according to the invention are all chemical compounds capable of transferring alkyl groups. Preference is given to chloromethane, bromomethane, iodomethane, dimethyl sulphate and dimethyl carbonate. Particular preference is given to dimethyl sulphate and chloromethane.

The alkoxyamines of the formula (V) and their acid addition complexes furthermore required as starting materials for carrying out reaction step d) of the process according to the invention are commercial chemicals for synthesis. Preferred acid addition complexes are the hydrochlorides, hydrogen sulphates or sulphates.

The invention also provides a process for preparing compounds of the formula (V)

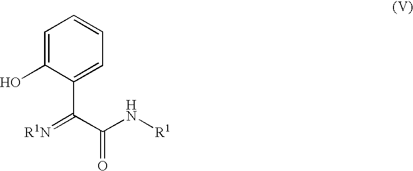

in which

R¹ represents C₁-C₄-alkyl,

characterized in that

a) a compound of the formula (II)

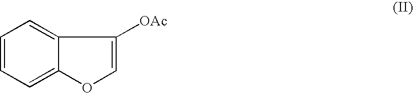

is reacted with chlorine gas, if appropriate in the presence of a diluent, and

b) the resulting compound of the formula (III)

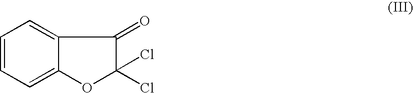

is reacted with an amine of the formula (IV),

R¹—NH₂ (IV),

in which

R¹ is as defined above

—or an acid addition complex thereof—,

if appropriate in the presence of a diluent and if appropriate in the presence of an acid acceptor.

The invention also provides the novel compounds of the general formula (I)

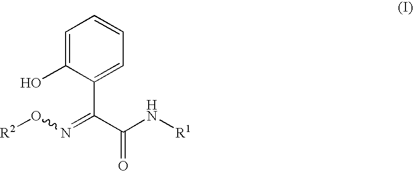

in which

R¹ represents C₁-C₄-alkyl and

R² represents C₂-C₄-alkyl.

In the novel compounds of the formula (I) R² preferably represents ethyl oder n-propyl; with particular preference, R² represents ethyl.

Reaction Step a)

Suitable diluents for carrying out reaction step a) of the process according to the invention are halogenated hydrocarbons, such as, by way of example and by way of preference, chlorobenzene, dichlorobenzene, dichloromethane, chloroform, carbon tetrachloride, dichloroethane or trichloroethane; nitriles, such as, by way of example and by way of preference, acetonitrile, propionitrile, n- or i-butyronitrile or benzonitrile; glacial acetic acid; esters, such as by way of example and by way of preference, methyl acetate or ethyl acetate; or alcohols, such as by way of example and by way of preference, methanol, ethanol, n- or i-propanol, n-, i-, sec- or tert-butanol, ethanediol, propane-1,2-diol, ethoxyethanol, methoxyethanol, diethylene glycol monomethyl ether, diethylene glycol monoethyl ether, mixtures thereof with water or pure water. In reaction step a), the diluent used is preferably glacial acetic acid.

When carrying out reaction step a) of the process according to the invention, the reaction temperatures can be varied within a relatively wide range. In general, the reaction step is carried out at temperatures of from 15° C. to 80° C., preferably at temperatures of from 15° C. to 60° C.

Reaction step a) of the process according to the invention is generally carried out under atmospheric pressure. However, it is also possible to operate under elevated or reduced pressure—in general between 0.1 bar and 10 bar.

For carrying out reaction step a) of the process according to the invention for preparing the compounds of the formula (I) in general from 2 to 5 mol, preferably from 2 to 3 mol, of chlorine are employed per mole of the compound of the formula (II).

Reaction step a) of the process according to the invention for preparing the compounds of the formula (I) is generally carried out as follows: the compound of the formula (II) (3-acetoxybenzofuran) is initially charged in the diluent, in particular in glacial acetic acid, and chlorine is added. The mixture is stirred, if appropriate at elevated temperature, until the reaction has gone to completion. After the reaction has ended, the mixture is worked up in a customary manner.

Reaction Step b)

Suitable diluents for carrying out reaction step b) of the process according to the invention are ethers, such as, by way of example and by way of preference, diethyl ether, diisopropyl ether, methyl t-butyl ether, methyl t-amyl ether, dioxane, tetra-hydrofuran, 1,2-dimethoxyethane, 1,2-diethoxyethane or anisole, or alcohols, such as by way of example and by way of preference, methanol, ethanol, n- or i-propanol, n-, i-, sec or tert-butanol, ethanediol, propane-1,2-diol, ethoxyethanol, methoxyethanol, diethylene glycol monomethyl ether, diethylene glycol monoethyl ether, mixtures thereof with water or pure water.

Reaction step b) of the process according to the invention is, if appropriate, carried out in the presence of a suitable acid acceptor. These include, by way of example and by way of preference, alkaline earth metal or alkali metal hydroxides, carbonates or bicarbonates, such as, for example, sodium hydroxide, potassium hydroxide, sodium acetate, potassium acetate, sodium carbonate, potassium carbonate, potassium bicarbonate or sodium bicarbonate.

When carrying out reaction step b) of the process according to the invention, the reaction temperatures can be varied within a relatively wide range. In general, the reaction step is carried out at temperatures of from 15° C. to reflux temperature, preferably at temperatures of from 20° C. to 40° C.

Reaction step b) of the process according to the invention is generally carried out under atmospheric pressure. However, it is also possible to operate under elevated or reduced pressure—in general between 0.1 bar und 10 bar and 10 bar.

For carrying out reaction step b) of the process according to the invention for preparing the compounds of the formula (I), in general from 4 to 10 mol, preferably from 4 to 8 mol, of the amine of the formula (IV) are employed per mole of the compound of the formula (III).

Reaction step b) of the process according to the invention is generally carried out as follows: the alkylamine of the formula (IV) is added to the compound of the formula (III), preferably in the presence of a diluent. The starting materials are stirred in a diluent, if appropriate at an elevated temperature, until the reaction has gone to completion. After the reaction has ended, the mixture is worked up in a customary manner.

Reaction Steps c1) and d)

Suitable diluents for carrying out reaction steps c1) and d) of the process according to the invention are all inert organic solvents. These include, by way of example and by way of preference, ethers, such as diethyl ether, diisopropyl ether, methyl t-butyl ether, methyl t-amyl ether, dioxane, tetrahydrofuran, 1,2-dimethoxyethane, 1,2-diethoxyethane or anisole; nitriles, such as acetonitrile, propionitrile, n- or i-butyronitrile or benzonitrile; amides, such as N,N-dimethylformamide, N,N-dimethylacetamide, N-methylformanilide, N-methylpyrrolidone or hexamethyl-phosphoric triamide; esters, such as methyl acetate or ethyl acetate; sulphoxides, such as dimethyl sulphoxide; sulphones, such as sulpholane; alcohols, such as methanol, ethanol, n- or i-propanol, n-, i-, sec- or tert-butanol, ethanediol, propane-1,2-diol, ethoxyethanol, methoxyethanol, diethylene glycol monomethyl ether, diethylene glycol monoethyl ether. Preferred diluents are alcohols, mixtures thereof with water or pure water.

Reaction steps c1) and d) of the process according to the invention are, if appropriate, carried out in the presence of a buffer medium. Suitable buffer media are all customary acid/salt mixtures which buffer the pH in the range from 1 to 7. Preference is given to using the mixture acetic acid/sodium acetate or no buffer medium.

When carrying out the reaction steps c1) and d) of the process according to the invention, the reaction temperatures can be varied within a relatively wide range. In general, the reaction steps are carried out at temperatures of from −20° C. to 150° C., preferably at temperatures of from 0° C. to 80° C.

Reaction steps c1) and d) of the process according to the invention are generally carried out under atmospheric pressure. However, it is also possible to operate under elevated or reduced pressure—in general between 0.1 bar and 10 bar.

For carrying out reaction step c1) of the process according to the invention for preparing the compounds of the formula (VI), in general from 1 to 15 mol, preferably from 1 to 2 mol, of hydroxylamine or an acid addition complex thereof are employed per mole of the compound of the formula (V).

For carrying out reaction step d) of the process according to the invention for preparing the compounds of the formula (I), in general from 1 to 15 mol, preferably from 1 to 2 mol, of alkoxyamine of the formula (VIII) or an acid addition complex thereof are employed per mole of the compound of the formula (V).

Reaction step c1) of the process according to the invention is generally carried out as follows: the compound of the formula (V) is, preferably in the presence of a diluent, admixed with hydroxylamine or its acid addition complex and, if appropriate, the buffer medium and heated. After the reaction has ended, the mixture is worked up in a customary manner.

Reaction step d) of the process according to the invention is generally carried out as follows: the compound of formula (V) is, preferably in the presence of a diluent, admixed with alkoxyamine or its acid addition complex and, if appropriate, the buffer medium and heated. After the reaction has ended, the mixture is worked up in a customary manner.

Reaction Step c2)

Suitable diluents for carrying out reaction step c2) of the process according to the invention are all inert organic solvents. These include, by way of example and by way of preference, aliphatic, alicyclic or aromatic hydrocarbons, such as, for example, petroleum ether, hexane, heptane, cyclohexane, methylcyclohexane, benzene, toluene, xylene or decalin; halogenated hydrocarbons, such as, for example, chlorobenzene, dichlorobenzene, dichloromethane, chloroform, carbon tetrachloride, dichloroethane or trichloroethane; ethers, such as, for example, diethyl ether, diisopropyl ether, methyl t-butyl ether, methyl t-amyl ether, dioxane, tetrahydrofuran, 1,2-dimethoxyethane, 1,2-diethoxyethane or anisole; ketones, such as, for example, acetone, butanone, methyl isobutyl ketone or cyclohexanone; nitriles, such as, for example, acetonitrile, propionitrile, n- or i-butyronitrile or benzonitrile; amides, such as N,N-dimethylformamide, N,N-dimethylacetamide, N-methylformanilide, N-methylpyrrolidone or hexamethylphosphoric triamide; esters, such methyl acetate or ethyl acetate; sulphoxides, such as dimethylsulphoxide; sulphones, such as sulpholane; alcohols, such as methanol, ethanol, n- or i-propanol, n-, i-, sec- or tert-butanol, ethanediol, propane-1,2-diol, ethoxyethanol, methoxyethanol, diethylene glycol monomethyl ether, diethylene glycol monoethyl ether, mixtures thereof with water or pure water.

Reaction step c2) of the process according to the invention is, if appropriate, carried out in the presence of a suitable acid acceptor. Suitable acid acceptors are all customary inorganic or organic bases. These include, by way of example and by way of preference, alkaline earth metal or alkali metal hydroxides, alkoxides, acetates, carbonates or bicarbonates, such as, for example, sodium methoxide, sodium ethoxide, potassium tert-butoxide, sodium hydroxide, potassium hydroxide, sodium acetate, potassium acetate, sodium carbonate, potassium carbonate, potassium bicarbonate or sodium bicarbonate, and also tertiary amines, such as, for example, trimethylamine, triethylamine, tributylamine, N,N-dimethylaniline, N,N-dimethyl-benzylamine, pyridine, N-methylpiperidine, N-methylmorpholine, N,N-dimethyl-aminopyridine, diazabicyclooctane (DABCO), diazabicyclononene (DBN) or diazabicycloundecene (DBU). In process step c2) preference is given to using potassium carbonate, sodium hydroxide or potassium hydroxide.

Reaction step c2) of the process according to the invention is, if appropriate, carried out in the presence of a suitable phase-transfer catalyst. These include, by way of example and by way of preference, quaternary ammonium salts, such as, for example, tetrabutylammonium bromide, chloride, hydrogensulphate or sulphate, methyltrioctyl ammonium bromide or chloride, hydrogensulphate or sulphate or 4-dimethylamino-N-(2-ethylhexyl)pyridinium bromide or chloride, hydrogensulphate or sulphate, quaternary phosphonium salts, such as, for example, tributyltetradecylphosphonium bromide or chloride, tetraphenylphosphonium bromide or chloride, crown ethers, such as, for example, dibenozo-18-crown-6, guanidiniumsalts, such as, for example, hexaalkylguanidinium chloride, and also polyethylene glycol derivatives. In process step c2), preference is given to using tetrabutylammonium bromide or tetrabutylammonium chloride.

When carrying out reaction step c2) of the process according to the invention, the reaction temperatures can be varied within a relatively wide range. In general, the reaction step is carried out at temperatures of from −20° C. to reflux temperature, preferably at temperatures of from 0° C. to 60° C.

Reaction step c2) of the process according to the invention is generally carried out under atmospheric pressure. However, it is also possible to operate under elevated or reduced pressure—in general between 0.1 bar and 10 bar.

For carrying out reaction step c2) of the process according to the invention for preparing the compounds of the formula (I), in general from 1 to 15 mol, preferably from 1 to 2 mol, of alkylating agent of the formula (VII) are employed per mole of the compound of the formula (VI).

Reaction step c2) of the process according to the invention is generally carried out as follows: the compound of the formula (VI) is, preferably in the presence of a diluent, admixed with a base and, if appropriate, a phase-transfer catalyst. The alkylating agent of the formula (VII) is added and the mixture is, if appropriate at elevated temperature, stirred until the reaction has gone to completion. After the reaction has ended, the mixture is worked up in a customary manner.

The examples below serve to illustrate the invention. However, the invention is not limited to the examples.

### PREPARATION EXAMPLES

### Example 1

2,2-Dichloro-1-benzofiran-3 (2H)-one

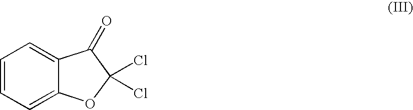

Reaction Step a)

At 50° C., 5.0 g of 3-acetoxybenzofuran are initially charged in 15 ml of glacial acetic acid, and 5.0 g of chlorine are introduced over a period of 1.5 hours. The mixture is stirred at 50° C. for another 0.5 hours and the solvent is then distilled off. The residue is taken up in toluene and, under reduced pressure, evaporated to dryness. This gives 5.7 g of 2,2-dichloro-1-benzofuran-3(2H)-one (GC: 98.1%; Yield: 98.4% of theory) as a solid.

(2Z)-2-(2-Hydroxyphenyl)-2-(methoxyimino)-N-methylethanamide:

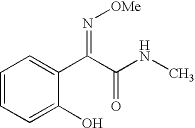

Process Steps b) and d) (One-Pot Process)

5.39 g of 2,2-Dichloro-1-benzofuran-3(2H)-one (GC: 97.9%) are initially charged in 60 ml of methyl tert-butyl ether, and 10.09 g of 40% strength aqueous methylamine solution are added at room temperature over a period of 10 minutes. The mixture is stirred for another 3 hours, and a solution of 8.8 ml of water, 2.61 g of methoxyamine hydrochloride and 2.56 g of sodium acetate is added at room temperature over a period of 5 minutes. The mixture is stirred for another 9 hours, the solvent is distilled off under reduced pressure and 150 ml of ice-water are added to the residue. The product is filtered off with suction, washed with water and dried at 50° C. under reduced pressure overnight. This gives (2Z)-2-(2-hydroxyphenyl)-2-(methoxyimino)-N-methylethanamide as a solid (4.6 g, HPLC-STD: 91.5%, 77.8% of theory).

### Example 2

2,2-Dichloro-1-benzofuran-3(2H)-one

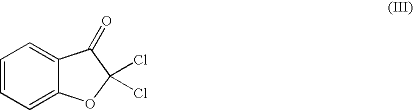

Reaction Step a)

At 50° C., 5.0 g of 3-acetoxybenzofuran are initially charged in 15 ml of glacial acetic acid, and 5.0 g of chlorine are introduced over a period of 1.5 hours. The mixture is stirred at 50° C. for another 0.5 hours and the solvent is then distilled off. The residue is taken up in toluene and, under reduced pressure, evaporated to dryness. This gives 5.7 g of 2,2-dichloro-1-benzofuran-3(2H)-one (GC: 98.1%; Yield: 98.4% of theory) as a solid.

(2Z)-2-(Hydroxyimino)-2-(2-hydroxyphenyl)-N-methylethanamide:

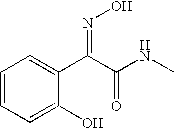

Process Steps b) and c1)

5.36 g of 2,2-dichloro-1-benzofuran-3(2H)-one (GC: 98.5%) are suspended in 14 ml of methanol, and 10.09 g of a 40% strength methanolic methylamine solution are added at room temperature over a period of 15 minutes. The mixture is stirred for another hour, and a solution of 5.0 ml of water and 2.59 g of hydroxylammonium sulphate is added at room temperature over a period of 10 minutes. The mixture is stirred for another hour, the solvent is distilled off under reduced pressure, water is added to the residue and the pH of the mixture is adjusted to 5. The mixture is extracted three times with ethyl acetate and the combined organic extracts are dried over sodium sulphate and evaporated to dryness. This gives (2Z)-2-(hydroxyimino)-2-(2-hydroxyphenyl)-N-methylethanamide as a solid (4.2 g, HPLC-STD: 85.2%, Z/E=81/19, 70.9% of theory).

### Example 3

2-(2-Hydroxyphenyl)-N-methyl-2-(methylimino)ethanamide:

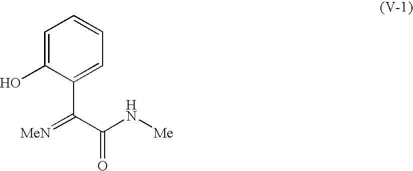

Reaction Step b)

5.0 g of 2,2-dichloro-1-benzofuran-3(2H)-one are initially charged in 20 ml of methyl tert-butyl ether, and 14.0 g of methylamine (41% strength in water) are added at room temperature over a period of 30 minutes. The mixture is stirred at room temperature for another 4.5 hours and then extracted three times with ethyl acetate, the combined organic extracts are washed with water, the organic phase is dried with sodium sulphate and the solvent is distilled off. This gives 4.4 g of 2-(2-hydroxyphenyl)-N-methyl-2-(methylimino)-ethanamide (GC: 93.0%; Yield: 86.5% of theory) as a solid.

What is claimed is:

1-11. (canceled)

12. A process for preparing compounds of formula (I) 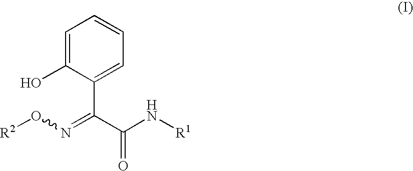
in which R¹ and R² represent C₁-C₄-alkyl, comprising
(a) reacting a compound of formula (II) 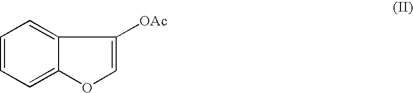
in which Ac represents acetyl,
with chlorine gas, optionally in the presence of a diluent, giving the compound of formula (III) 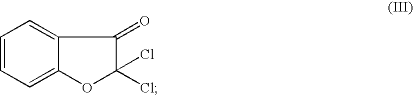
(b) reacting the resulting compound of formula (III)
with an amine of formula (IV)
R¹—NH₂ (IV)
in which R¹ is as defined for formula (I),
or an acid addition complex thereof,
optionally in the presence of a diluent and optionally in the presence of an acid acceptor, giving a compound of formula (V) 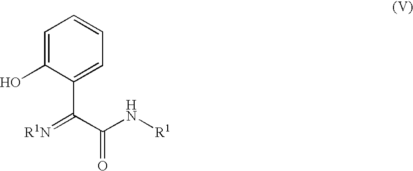
in which R¹ is as defined for formula (I);
(c1) reacting the compound of formula (V) with hydroxylamine or an acid addition complex thereof, optionally in the presence of a diluent and optionally in the presence of a buffer medium, giving a compound of formula (VI) 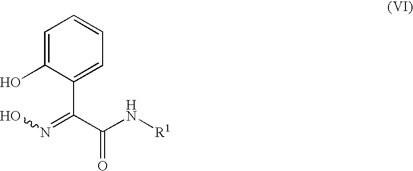
in which R¹ is as defined for formula (I); and
(c2) reacting the compound of formula (VI) with an alkylating agent of formula (VII)
R²_X (VII)
in which
X represents halogen, —O—CO—O—R², —O—SO₂—R², or —O—SO₂—O—R², and
R² is as defined for formula (I),
optionally in the presence of a diluent, optionally in the presence of an acid acceptor, and optionally in the presence of a phase-transfer catalyst;
or
(d) reacting the compound of formula (V) with an alkoxy amine of formula (VIII)
R²—O—NH₂ (VII),
in which R² is as defined for formula (I),
or an acid addition complex thereof,
optionally in the presence of a diluent, optionally in the presence of an acid acceptor, and optionally in the presence of a buffer medium.

13. A process according to claim 12 in which reaction steps (a) and (b) are carried out at from 20° C. to reflux temperature.

14. A process according to claim 12 in which the diluent used in reaction step (a) is glacial acetic acid.

15. A process according to claim 12 in which R¹ and R² represent methyl.

16. A process according to claim 12 in which from 2 to 5 mol of chlorine, based on the compound of the formula (II), are used in reaction step (a).

17. A process according to claim 12 in which from 4 to 8 mol of the alkylamine of formula (IV), based on the compound of the formula (III), are used in reaction step (b).

18. A process according to claim 12 in which the amine of formula (IV) used in reaction step (b) is methylamine.

19. A process according to claim 12 in which reaction steps (b) and (d) are carried out as a one-pot process.

20. A process according to claim 12 in which reaction steps (b) and (c1) are carried out as a one-pot process.

21. A process for preparing compounds of formula (V) 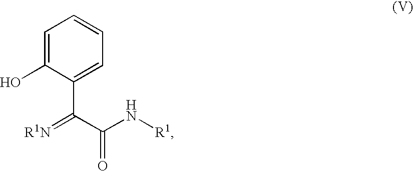
in which R¹ represents C₁-C₄-alkyl, comprising
(a) reacting the compound of the formula (II) 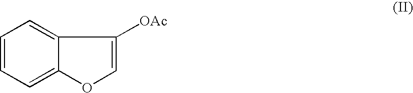
in which Ac represents acetyl,
with chlorine gas, optionally in the presence of a diluent, giving the compound of formula (III) 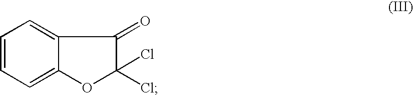
(b) reacting the resulting compound of formula (III) with an amine of formula (IV)
R¹—NH₂ (IV)
in which R¹ is as defined for formula (I),
or an acid addition complex thereof,
optionally in the presence of a diluent and optionally in the presence of an acid acceptor.

22. A compound of formula (I) 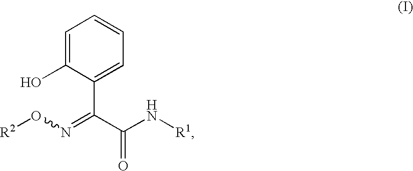
in which
R¹ represents C₁-C₄-alkyl and
R² represents C₂-C₄-alkyl.
